(19) United States
(12) Patent Application Publication
(10) Pub. No.: US 2019/0358069 A1
(43) Pub. Date: Nov. 28, 2019

(54) SLEEVE TUBE AND METHOD OF USE

(71) Applicant: Kent C. Sasse, (US)

(72) Inventors: Kent C. Sasse, Reno, NV (US); Matthew T. Fisher, Reno, NV (US)

(73) Assignee: Kent C. Sasse, Reno, NV (US)

(21) Appl. No.: 16/538,547

(22) Filed: Aug. 12, 2019

Related U.S. Application Data
(63) Continuation of application No. 15/332,897, filed on Oct. 24, 2016, now abandoned.
(60) Provisional application No. 62/245,524, filed on Oct. 23, 2015.

(51) Int. Cl. A61F 5/00; A61B 5/00; A61M 1/00; A61M 25/10

(52) U.S. Cl. CPC A61F 5/0089; A61B 5/4238; A61M 1/0039; A61M 2205/0266; A61M 2210/105; A61M 2210/1053; A61M 25/10

(57) ABSTRACT

Methods, systems, and devices are described for fabricating and using an orogatric tube. The orogastric tube may have: a proximal end section; a distal end section opposite the proximal end section, the distal end having a flexible, resilient curved portion; at least one sump channel extending from the proximal end section along a pre-determined length of the orogastric tube to the distal end section; at least one balloon channel extending from the proximal end section along a pre-determined length of the orogastric tube to the distal end section, the balloon channel being in communication with an expandable balloon in the distal end section; and a main channel enclosing a pre-determined length of both the sump channel and the balloon channel.

### CROSS-REFERENCE TO RELATED APPLICATIONS

This application is a continuation of applicants' prior U.S. patent application Ser. No. 15/332,897, filed Oct. 24, 2016, and titled “Sleeve Tube and Method of Use”, which claims priority to, and incorporates by reference, the applicants' prior provisional patent application, titled Sleeve Tube and Method of Use, Ser. No. 62/245,524, filed Oct. 23, 2015. All such prior applications are hereby incorporated by reference in their entirety. It is to be understood, however, that in the event of any inconsistency between this specification and any information incorporated by reference in this specification, this specification shall govern.

### FIELD OF THE DISCLOSURE

The present disclosure relates to an orogastric sleeve tube (“sleeve tube”) and methods of use, and in one aspect relates to a multi-channel sleeve tube that may be used in laparoscopic sleeve gastrectomy surgery. The sleeve tube may combine unique features of a gastric aspiration tube utilizing sump technology in combination with a novel sizing calibration tube.

In some embodiments, the sleeve tube may be a calibration device to be used within the lumen of a patient's stomach, where the sleeve tube may be fabricated with a material or a combination of materials enabled to provide a modifiable curvature which can conform to the natural shape of the stomach. In other embodiments, sump aspiration may be enabled to evacuate gastric contents from the stomach. The sleeve tube further provides a unique diameter augmentation balloon at the modifiable curvature of the sleeve tube and/or incisura area of the stomach (e.g., an angular notch in the stomach indicative of a separation point between the body of the stomach and where the stomach connects to the duodenum). In still other embodiments, the sleeve tube may provide a mechanism to perform a leak test after the completion of a sleeve gastrectomy.

In some embodiments, the sleeve tube may be used for gastrectomy surgeries for gastric tumors, and/or for other types of procedures such as, but not limited to, laparoscopic sleeve gastrectomy surgery, bariatric surgery, and endoscopic procedures.

### BACKGROUND OF SOME ASPECTS OF THE DISCLOSURE

Sleeve gastrectomy surgery is one of the most frequently performed procedures for the treatment of morbid obesity with estimates that more than 200,000 gastrectomy procedures may be performed in the United States in the next year. Using existing technology, operating room personnel use multiple individual tubes to perform gastric procedures. The multiple individual tubes include (i) a standard tube with sump feature to evacuate the gastric contents, such as a nasogastric tube (ii) a sizing calibration tube during the cutting/stapling of the stomach, and (iii) another insertion of a standard nasogastric tube at the end of the procedure to instill colored dye and distend the stomach for a leak test.

Existing tube technology fails to conform to the curvature of the natural human stomach, and thus creates risks of technical complications in sleeve gastrectomy surgery—the existing tubes are typically straight, having no curvature and no way to make diameter adjustments at the incisura. Existing straight gastrectomy calibration system tubes include, for example, a ViSiGi 3D tube from Boheringer Ingelheim (e.g., having a French (Fr) gauge of 36 or 40). These tubes are used to decompress the stomach and/or remove gastric fluids with suction. In addition, these tubes may be used for irrigation and/or as a sizing guide during surgery; however, the Boehringer Ingelheim tubes do not have adjustable curvature, lack a sump channel, and lack both a balloon channel and a balloon to augment the diameter at the incisura. Other existing technology includes standard nasogastric tubes that lack a sump channel, and thus a separate sump tube must be used such as a Nasogastric Sump tube model number 0042140 from Bard Medical (for example, shaving French gauges of 10 Fr, 12 Fr, 14 Fr, 16 Fr, and 18 Fr).

Furthermore, existing tube technology fails to suction, aspirate, and deflate the stomach adequately; commonly used prior art tubes either have no suction capabilities or the tubes use a simple, single-channel suction system that does not involve a sump, resulting in a system that can easily become clogged with gastric mucus or particulate matter. In addition, existing technology may employ a tube having a blunt tip, where the tip is difficult to insert into the patient's stomach or results in anatomical trauma.

### BRIEF SUMMARY OF SOME ASPECTS OF THE DISCLOSURE

The applicants believe they have discovered at least some of the problems and issues with the prior art noted above. They have therefor invented a multi-channeled sleeve tube sleeve for use in gasterectomies and other procedure. Other procedures may include, for example, surgeries for gastric tumors, bariatric surgery, and endoscopic procedures and other surgeries where a curved calibration tube can be utilized. Yet other procedures may be performed as described infra.

One aspect of the present disclosure provides a multi-channeled sleeve tube having two or more among a main channel, a sump channel, and a balloon channel extending through a body section of the sleeve tube. In some embodiments, the sleeve tube of the present disclosure combines many of the features of multiple, independent tubes of the prior art, while also providing, in some embodiments, a curved working section adjacent the distal end of the sleeve tube.

In some embodiments, having one multi-channeled tube that performs multiple functions can eliminate or reduce the number of placements and removals of tubes into the patient's esophagus; and in some applications, reducing the number of placements can be particularly useful because each time a tube is introduced there is a risk of perforation, laceration, and injury to the tissues of the oropharynx and esophagus. By eliminating two or three passage procedures, in at least a substantial number of applications, complications to the patient can be significantly decreased, and cost savings can accrue because valuable operating room time can be saved.

In some embodiments, the distal end of the sleeve tube may have a series of perforations that may, in some instances, further enable evacuation of gastric contents as well as facilitate injection of fluid into the stomach such as during a dye leak test. Additionally, some embodiments of the sleeve tube of the present disclosure may provide a sump channel to vent or supply air or gas when using the sleeve tube.

Some embodiments have a balloon channel coupled to a balloon mounted on the distal end section of the sleeve tube. In some embodiments, the diameter of the balloon may be adjusted by increasing the volume of air or gas forced into the balloon. In some embodiment, inflation of the balloon may cause the working section of the tube sleeved to curve or further curve.

In some embodiments, the sleeve tube of the present disclosure can include a soft, tapered distal nose or tip that can, in some applications, facilitate smoother and less traumatic insertion of the sleeve tube into the patient's mouth, esophagus, and stomach, reducing the incidence of sore throat, tearing of the esophageal lining, and esophageal bleeding.

In some embodiments having a curved or curvable working section, the curvature can established using a plurality of thermoplastic materials having “shape memory” properties that cause the working section to be biased toward providing a free-state predetermined curvature at certain temperatures, such as the internal temperature of the lumen of the stomach, and to be biased toward be straight in the free state at normal room temperature. In some applications, when the sleeve tube is inserted into the stomach, the temperature of the stomach causes the working section to curve to conform more closely to the natural curvature of the interior stomach wall.

Other embodiments may provide a sleeve tube with a flexible, resilient working section permanently biased to a predetermined curved free state. The working section can easily straighten for insertion or withdrawal through the patient's esophagus while returning to the curved state in the patient's stomach.

Other advantages of various embodiments of the sleeve tube can variously include the reduction in complications such as leaks, stenosis, obstruction, and/or encroachment at the incisura which results in improved patient outcomes, decreased complications, and reduction in costs that would otherwise be incurred during the corrective procedures and subsequent medical care.

Obstruction of the sleeve can occur in approximately 1% of sleeve gastrectomy procedures due to a phenomenon termed the “wind sock deformity” in which the lower stomach twists and folds, effectively blocking the channel through the stomach lumen. At least some embodiments of the sleeve tube can prevent the “wind sock deformity” complication by maintaining a natural anatomic curve of the stomach and preventing the surgeon from introducing a twist in the gastric sleeve that could favor a folding event that would create obstruction.

In addition, post-operative stenosis or narrowing occurs in between 1% and 3% of cases of sleeve gastrectomy. These cases are then typically treated with endoscopic dilatation procedures in the weeks and months following the initial surgery, at considerable expense. At least some embodiments of the sleeve tube described in this disclosure can solve this problem by adding volume to the calibration tube at the most vulnerable area, thus creating additional space and area where narrowing might otherwise occur.

This disclosure provides a novel system and method of fabrication and use of a multi-channeled sleeve tube. There are many other novel features and aspects of this disclosure. The will become apparent as this specification proceeds. It is to be understood, however, that the scope of a claim in this matter is to be determined by the claim as issued and not by whether the claim addresses an issue, or provides a feature, because the issue or feature is referenced in the Background or Brief Summary sections above.

### BRIEF DESCRIPTION OF THE DRAWINGS

The applicants' preferred and other embodiments are described in association with the accompanying Figures in which:

FIG. 1 is an elevational view of a sleeve tube;

FIG. 2 is a partial cross-sectional view taken along section line A-A of FIG. 1;

FIG. 3A is an elevational view of an aperture section mounted to the main body section of the sleeve tube of FIG. 1;

FIG. 3B is a cross-sectional view of the structure of FIG. 3A;

FIG. 4A is a partial cross-sectional view taken along section line A-A of FIG. 1, showing an inflatable balloon in an inflated state;

FIG. 4B is a partial cross-sectional view of the structure of FIG. 4A with the balloon in the deflated state;

FIG. 5 is a cross-sectional view taken along section line D-D of FIG. 1, showing a section of a coupler and multiple channels of the sleeve tube of FIG. 1;

FIG. 6A is a cross-sectional view of an alternative structure for the multiple channels in a sleeve tube;

FIG. 6B is a cross-sectional view of a yet further structure providing multiple channels in a sleeve tube;

FIG. 7A is a cross-sectional view taken along section line B-B of FIG. 1, showing the balloon in an inflated state;

FIG. 7B is a cross-sectional view of the structure of FIG. 7A with the balloon in a partially deflated state

FIG. 8A is a partial elevational view of a section of the sleeve tube of FIG. 1 adjacent the sleeve tube's distal end;

FIG. 8B is a partial elevational view showing an alternative balloon shape provided adjacent an alternative sleeve tube's distal end;

FIG. 9 shows an exploded perspective view of the sleeve tube of FIG. 1;

FIG. 10 is a front elevational view of an alternative embodiment of a sleeve tube;

FIG. 11 is a partial cross-sectional view, taken along section line E-E of FIG. 10, of the aperture section secured to the main body section;

FIG. 12A is a side elevational view of the sleeve tube of FIG. 10;

FIG. 12B is a side elevational view of the sleeve tube structure of FIG. 12A with the balloon inflated to cause further curvature of the working end of the sleeve tube;

FIG. 13 is a partial side elevational view of the sleeve tube of FIG. 10 with the balloon inflated;

FIG. 14 is cross-sectional view taken along section line F-F of FIG. 10;

FIG. 15 is a cross-sectional view taken along section line G-G of FIG. 10;

FIG. 16A is a cross-sectional view taken along section line H-H of FIG. 10, showing the balloon in the inflated state;

FIG. 16B is a cross-sectional view of the structure of FIG. 16A but with the balloon deflated;

FIG. 17A is an elevational view of the sleeve tube of FIG. 10 in a pre-curved or otherwises straightened state;

FIG. 17B is an elevational view of the sleeve tube of FIG. 17A in a partially curved state; and

FIG. 17C is an elevational view of the sleeve tube of FIG. 17B in a further curved state brought about by inflation of a balloon in the curved section.

### DETAILED DESCRIPTION OF SOME EMBODIMENTS

The prior Brief Summary and the following description provide examples that are not limiting of the scope of this specification. One skilled in the art would recognize that changes can be made in the function and arrangement of elements discussed without departing from the spirit and scope of the disclosure. Various embodiments can omit, substitute, add, or mix and match various procedures or components as desired. For instance, the methods disclosed can be performed in an order different from that described, and various steps can be added, omitted, or combined. Also, features disclosed with respect to certain embodiments can be combined in or with other embodiments as well as features of other embodiments.

In an exemplary gastrectomy procedure, one embodiment of the sleeve tube includes each of a balloon channel, main channel, and sump channel extending from the proximal end of the sleeve tube to the working, distal section of the sleeve tube. The distal end of the sleeve tube is inserted into a patient's and through the patient's esophagus into the patient's stomach. Laparoscopic technology can also be utilized to operation personnel to view the placement of the sleeve tube on video monitors present in the operating room. I

In some cases, the sleeve tube may be comprised of a low friction material to facilitate easier entry into the body. The sleeve tube may alternatively or in addition be lubricated with a water-soluble lubricant prior to the insertion step.

First, gastric contents are aspirated from within the lumen of the stomach through perforations in the working end of the sleeve tube and then through the main channel and possibly the sump channel as well.

The sleeve tube is then advanced into the stomach so that its distal tip passes along the lesser curvature aspect of the stomach. Operation personnel can adjust the working section of the sleeve tube to provide the proper placement, adjustment of the sleeve tube curvature, and inflation of the sleeve tube balloon as desired.

When the sleeve tube is in position, the balloon is inflated, the desired curvature is established in the working section, the operation personnel can then use conventional suction techniques to apply suction to the main suction tube to hold gastric tissues snugly to the tube so that the surgery may then proceed. Thus, the sleeve tube serves as a stomach sizing device, enabling the surgeon to remove the outer portion of the stomach safely.

After completion of the sleeve tube gastrectomy procedure, the surgeon can also use the sleeve tube to perform a leak test to test the integrity of the staple line on the stomach. A leak test can be done by injecting colored dye into the main channel of the multi-channeled sleeve tube while the surgeon occludes the sump outlet, thus tautly distending the stomach and stressing a newly created staple line. The surgeon can then observe the integrity of the staple line. When desired, the dye is aspirated through the main channel, the balloon is deflated, and the sleeve tube is then removed from the patient.

The sump channel can be used during the procedure to inject gas or air into the distal end section of the sleeve tube. The sump channel can thus aid to clear blockage of sleeve tube perforations and the main channel. The sump channel can also be used to prevent excessive sucking through the main channel and the associated perforations in communication with the main channel.

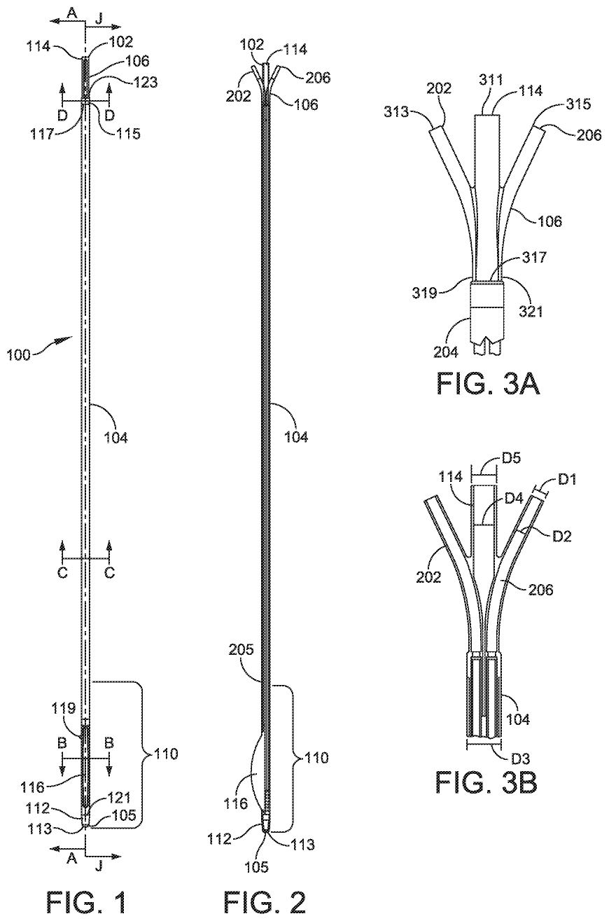

Referring now to FIG. 1, one embodiment an elongated sleeve tube 100 has a proximal end 102 opposite a distal end 105. The sleeve tube 100 can provide a multi-channel orogastric tube system for use in conducting sleeve gastrectomy surgery. In other embodiments, the sleeve tube 100 also or alternatively can be used as a calibration device within the lumen of a person's stomach.

The sleeve tube 100 may be conceptually divided into four sections: the elongated sleeve tube 100 as a whole; an aperture section 106 providing differing apertures for each three tubular channels or conduits; a main body section 104 secured to the distal end of the aperture section 106 and having three tubular channels extending longitudinally within and along the length of the sleeve tube 100; and an expandable section 110 extending from the main body section 204 at the distal end 105 of the sleeve tube 100. The expandable section 110 terminates in a tapered nose section 112 at the distal end 113 of the expandable section 110.

Aperture section 106 attaches to the main body section 204 by way of a tubular coupler end section 115 at the distal end 123 of the aperture section 106. In turn, the main body section 104 penetrates the distal end 117 of the coupler end section or sleeve 115 to couple the main body section 104 to the aperture section 106.

In some embodiments, a balloon 116 is mounted to, or formed in, the expandable section 110 of the sleeve tube 100. The balloon 116 can be controllably inflated outwardly from, and controllably deflated to retract toward, the body 119 of the expandable section 110.

The sleeve tube 100 can thus be formed of separate sections and elements, for example, 106, 104, 110, and 112, joined together by adhesives or other inter-connecting devices or methods (for example, by thermal bonding or fusing techniques). The adhesives can be, but are not limited to, Federal Drug Administration (FDA) approved medical adhesive materials (for example, Luer-Lok, Luer-Slip, catheter tip, barbed fittings, solvents, etc.). In other embodiments, the sleeve tube 100 may be molded (for example, by extrusion or injection molding) as a single piece without the need to join together multiple pieces. In yet other embodiments, the sleeve tube 100 or any of its components can be three-dimensionally printed using polymeric or other suitable material.

In one embodiment, the nose section 112 is first formed as a separate unit from the balance of the expandable section 110 and secured to the balance of the expandable section 110 with medical adhesive or other coupling devices or methods as described above. The nose section 112 is frustoconical, or somewhat so, to provide a conically narrowing but rounded distal end 113 of the nose section 112 opposite its junction with the balance of the expandable section 110. The proximal end 121 of the nose section 112 secured to the balance of the expandable section 110 is approximately the same diameter in width as the balance of the expandable section 110 (e.g., 13.3 mm or 40 Fr).

In some embodiments, the nose section 112 is made of the same material as the balance of the expandable section 110 (and may formed as part of it) to provide similar flexibility and resilience for the expandable section 110 and the nose section 112. In some embodiments, the nose section 112 provides an atraumatic distal end 105 that can help prevent mucosal trauma as the sleeve tube 100 is inserted into the patient's body.

With reference now to FIG. 2, the aperture section 106 has three separate channels or tubes 114, 202, 206 providing three corresponding tubular lumens (that is, cavities or passages), with these lumens extending from the aperture section 106 longitudinally through main body section 104 to penetrate the expandable section 110 of the sleeve tube 100. The expandable section 110 extends from the distal end 205 of the main body section 104 and includes an outwardly inflatable balloon 116, shown in an inflated state in FIG. 2.

With reference now to FIG. 3A, the tubular proximal ends 311, 313, 315 of the main channel 114, balloon channel 202, and sump channel 206, respectively can inter-connect with syringes, suction devices, or stopcocks as desired. The opposed ends 317, 319, 321 of the main channel 114, the balloon channel 202, and the sump channel 206, respectively, converge to penetrate the main body section 104 and provide a multi-channeled tubular main body section 204 having a tubular outer periphery surrounding the main channel 114, balloon channel 202, and sump channel 206 contained within the tubular outer periphery of main body section 104.

With reference to FIG. 3B, the outer diameter D1 of the balloon channel 202 is the same as the outer diameter of the sump channel 206, whereas the outer diameters D3 and D5 of the main channel 114 and main body section 104, respectively, are larger than D1. In yet other embodiments, the outer diameters of channels 202, 204, and 206 may otherwise differ or be the same as desired, and thus they may all differ from one another, respectively, if desired for a given application.

Similarly, the inner diameters (that is, the lumen diameters) of the balloon channel 202 and sump channel 206 may have the same, or approximately the same, diameter D2, including extending along and within the lateral length of the main body section 104. The inner diameter D4 of the main channel 114 may be larger than D2. In yet other embodiments, the inner diameters of channels 202, 204, and 206 may all differ from other another, respectively, or be similar as otherwise desired.

In one example, D3 may be between 28 Fr and 52 Fr, with a preferred diameter of 40 Fr. These exemplary diameters may be altered as desired.

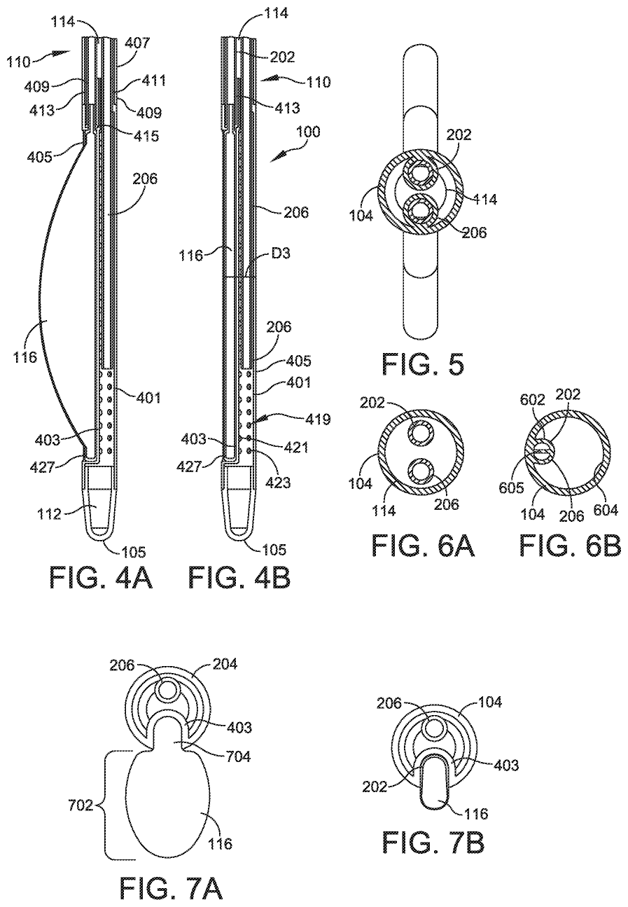

With reference to FIGS. 4A and 4B, the expandable section 110 has a central laterally extending, generally tubular section 401 extending from the main body section 104 and terminating in the nose section 112. The balloon 116 is securely mounted in a balloon mounting slot 403 penetrating, and laterally extending along, the outer periphery 405 of the central generally tubular section 401,

The distal end 407 of the main tube section 204 has a thinned wall 409 providing a female distal receptacle 409 to matingly surround, abut, and grasp (in conjunction with adhesive to form a secure bond with) a narrowed mating male proximal end 411 of the expandable section 110. The distal end 413 of the balloon tube 202 connects to the proximal end 415 of the balloon 116 so that the balloon tube 202 can thereby (i) inject air or other gas into the balloon 116, causing the balloon 116 (i) controllably inflate by injecting air or other gas in the proximal end (not shown in FIGS. 4A and 4B) of the balloon channel 202, and (ii) controllably deflate by venting or withdrawing air or other gas from the proximal end (id.) of the balloon channel 202 to, with reference to FIG. 4B, collapse the balloon 116 within the balloon mounting slot 403. In the deflated state, the outer periphery of the balloon 116 lies flush with the outside surface of the curved working section 110. In this deflated state, the sleeve tube 100 may be inserted through the patient's mouth and esophagus and into the patient's stomach.

With continuing reference to FIG. 4B, the sump channel 206 extends past the female distal receptacle 409 well into the general tubular interior of the generally tubular section 401. The generally tubular section 401 has multiple laterally extending rows, e.g., 417, 419, of tubular perforations, e.g., 421, 423, penetrating the generally tubular section 401 and extending from the interior to the outer periphery 405 of the generally tubular section 401. The sump channel 206 thus can be used to withdraw or inject gas into the generally tubular section 401, and the main channel 114, which is in communication with the interior of the generally tubular section 401, can inject or withdraw gas, and withdraw material from, the interior of the generally tubular section 401 through the perforations, e.g., 421, 423, in that section 401. Material or gas sucked into the generally tubular section 401 can be withdraw from the stomach through the main channel 114, and the sump channel 206 can be used to inject air or gas into the generally tubular section 401 in order to, for example, clear blockage of material within the perforations, e.g., 421, 423. The sump channel 206 can alternatively be used to aspirate gastric contents of the patient's stomach.

In contrast, a single channel tube system (for example, in procedures using multiple separate tubes) may clog easily, thus resulting in a failure to successfully evacuate gastric contents. The sump channel 206, as part of the multi-channel system 100, allows air to travel through the system and provides a secondary channel that may be used to clear a clogged tube, thus improving the effectiveness when the system is used as an evacuation tool for gastric contents. Furthermore, use of the sump channel 206 can be used to vent the main channel 114 reduce the risk of applying too much suction pressure to the main channel 114, resulting in gastric mucosal lining tissue being pulled toward and within perforations, e.g., 421, 423, in the sleeve tube 100, which can lead to tearing of the stomach lining or bleeding when the sleeve tube is removed.

The balloon 116 may be fabricated by dip forming of a thermoset polymer, or by blow molding or extrusion of a thermopolymer, such as polyvinyl chloride, polyurethane, etc. The balloon 116 may be affixed to the balloon mounting slot 403 with compatible medical adhesives, by heat shrinkable tubing, by mechanical means such as thread ties for example, or by a combination of such techniques and/or others.

In one embodiment, the uninflated width W1 of the inflatable section 110 is 10 mm and the lateral length of the balloon 116 is 6 cm, with the distal end 427 of the balloon 116 spaced 2 cm from the distal end 105 of the sleeve tube. When air or other gases are inserted into the balloon 116, the balloon inflates to cause the width of inflatable section to increase to, for example, 23.3 mm. The volume of the balloon can 116 vary of course, and in some embodiments, the balloon can fully inflate when pressurized with air or gas to 2 ATM. In addition, the balloon may be further pressurized to provide a more rigid balloon for greater support.

With reference now to FIG. 5, the balloon channel 202 and the sump channel 206 may be located inside and coupled to, or abutting, the interior side wall of the main body section 104. Alternatively, as shown in FIG. 6A, the balloon channel 202 and the sump channel 206 may be spaced from the interior side or wall of the main channel 204.

In another alternative of FIG. 6B, the main body section 204 includes an interior tube 602 extending inwardly from the interior surface 604 of the main tube 602. The portions of the balloon channel 202 and sump channel 206 within the main body section 104 can consist of separated, opposed D-shaped channels sharing a common central wall 605 within the interior tube 602. A laterally extending portion of the interior tube 602 penetrates, and is partially formed within, a portion of the wall of the main body section 104.

With reference now to FIGS. 7A and 7B, a static, secured portion 704 of the balloon 116 is secured to the interior wall of the portion balloon channel 202 within the balloon mounting slot 403 in the expandable section 110. As it is inflated, the balloon 116 extends outwardly from balloon mounting slot, and in the embodiment of FIG. 7A, the balloon 116 expands to provide an inflated section 702 having an oblong cross-section extending from, and along the lateral length of, the U-shaped balloon mounting slot 403.

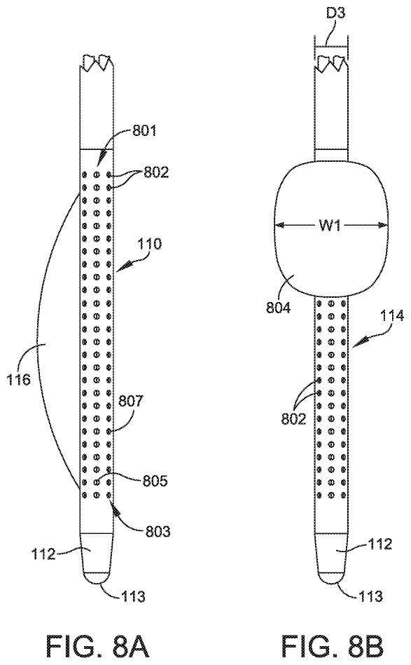

With reference to FIG. 8A, the parallel rows of perforations, e.g., 801, 803, extend laterally along, and all around the general tubular periphery of the bulk of the expandable section 11. In one embodiment, the rows of perforations, e.g., 801, 803, are located within 15-20 cm of, as shown in FIG. 1, the distal end 105 of the sleeve tube 100. The number, size, and location of the perforations, e.g., 805, 807, can be varied to facilitate removal of particulates when suction is applied to the main channel 204 (not shown in FIG. 8A). In addition, the perforations, e.g., 805, 807, can enable a leak test in which colored dye is forced into the sleeve tube 100 to fill the stomach through the perforations, e.g., 805, 807, to distend the stomach and test a surgical staple line for leakage

Referring now to FIG. 8B, an alternative embodiment of the expandable section 110 has a differing or additional balloon 804. This balloon 804 expands to provide a rounded exterior periphery substantially wider W3 than external diameter D3 of the balance of the laterally extending expandable section 110.

In another embodiment (not shown), yet another balloon structure, along with a supporting channel, can be also be included in a single sleeve tube to augment the diameter of the sleeve tube at, for example, just below the gastroesophageal junction. This location is an area where surgeons generally should avoid encroachment and making the stomach too tight, which can result in leaks and strictures.

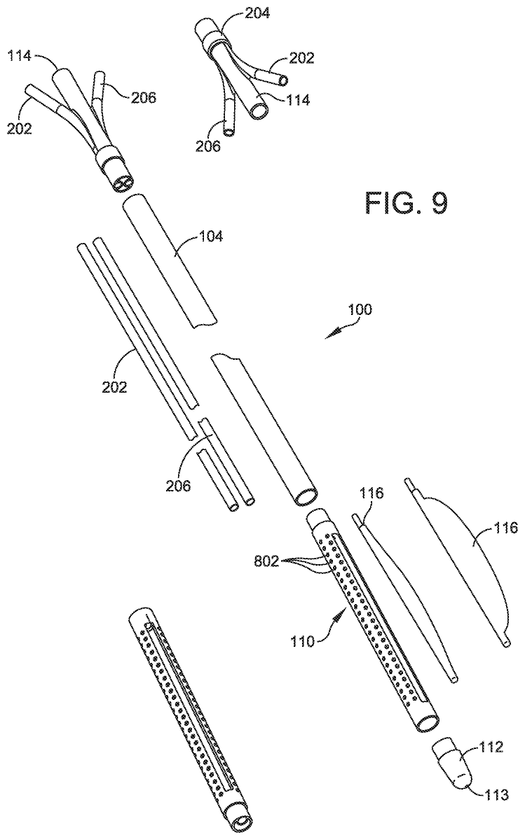

With reference now to FIG. 9, the sleeve tube 100, including any or all of the associated sections and parts, may be manufactured in any of many ways. In one example, each of the elements of sleeve tube 100 shown in FIG. 9 may be fabricated individually and then subsequently assembled into the completed sleeve tube 100. In some embodiments, the sleeve tube 100 may be molded in two halves (less the balloon) combined to form a complete device from a thermoset material (silicone for example). The balloon can then secured to the sleeve tube 100 with adhesive.

In another example, at least the main body section 104 may be injection molded using a thermoset material (silicon for example), with each of three channels 202, 204, and 206 created by inserting long core pins that are removed after the entire sleeve tube assembly is removed from the mold.

In yet another example, elements of the sleeve tube 100 assembly such as shown in FIG. 9 may be extruded or molded separately of silicone rubber. More specifically, the sleeve tube's main body section 204 can be formed of extruded silicone rubber formed with a distal end curvature before vulcanizing. The branching aperture end 106 can be injection molded. The expandable section 110 can be injection molded to provide the perforations 802 and balloon mounting slot 403 (or other balloon mounting structure), and nose section 112. After production, the parts can be bonded together such as with adhesive or other bonding techniques well known in the art. Other methods of production may be utilized, such as three-dimensional printing for example.

In order to facilitate passage of the sleeve tube 100 into the stomach and to enable the creation of the adjustable curvature of the curvable working section 110, the sleeve tube 100 can be fabricated or coated with a low friction polymer, such as, but not limited to, polytetrafluoroethylene (PTFE) or other hydrophilic materials. In one embodiment, at least the working section 110 alternatively at least dominantly consists of silicone, with curvature of this section 100 formed a in a secondary curing process.

In some embodiments, each or any of the parts, sections, or elements described may be symmetrical along an axis; however, in other embodiments, the parts, sections, or elements may be asymmetrical. For example, a proximal end may be thicker than a distal end, or different materials may be used at one end versus another. In some cases, the material may be patterned in one section and not in others.

In one embodiment, the sleeve tube 100 is approximately 100 cm in length from the proximal ends of the tube at the aperture section 106 to the distal end 105 of the nose section 112 and has a diameter of approximately 13.3 mm (40 Fr). These dimensions may be adjusted as needed or desired for differing applications. Generally, however, the diameter of the sleeve tube 100 for human gastric applications may be up to 150% greater than 13.3 mm, and the length of such a sleeve tube may be up to 75% shorter and 100% longer than 100 cm.

When the sleeve tube 100 is properly placed within a patient's stomach, the balloon 116 may be inflated at a desired location within the stomach, such as at the gastric incisura or other desired locations causing them to similarly inflate. The ability to increase the diameter of the sleeve tube (and more specifically the curvable working section 110 of the sleeve tube 100) may result in improving the safety of the sleeve procedure and/or prevent complications resulting from stenosis, staple link leaks, or gastric obstruction.

A primary risk factor in the development of gastric staple line leaks is the development of narrowing or stenosis at the lower part of the sleeve, which then increases the intra-luminal pressure, causing leaks. Some embodiments prevent this occurrence through the inflation of balloon 116, which can add an additional up to 5-25 mm of width, and in one particular embodiment up to 10 mm of width, to the sleeve tube 100 at locations where stenoses typically form (for example, in the lower sleeve incisura region). After the sleeve procedure is completed, and the leak test is finished, the balloon 116 is deflated, and the sleeve tube 100 is removed from the patient.

In some embodiments, the sleeve tube 100 may be used for veterinarian applications. The lengths, diameters, and thicknesses, etc., of the sleeve tube 100 and corresponding components may be sized appropriately for such applications.

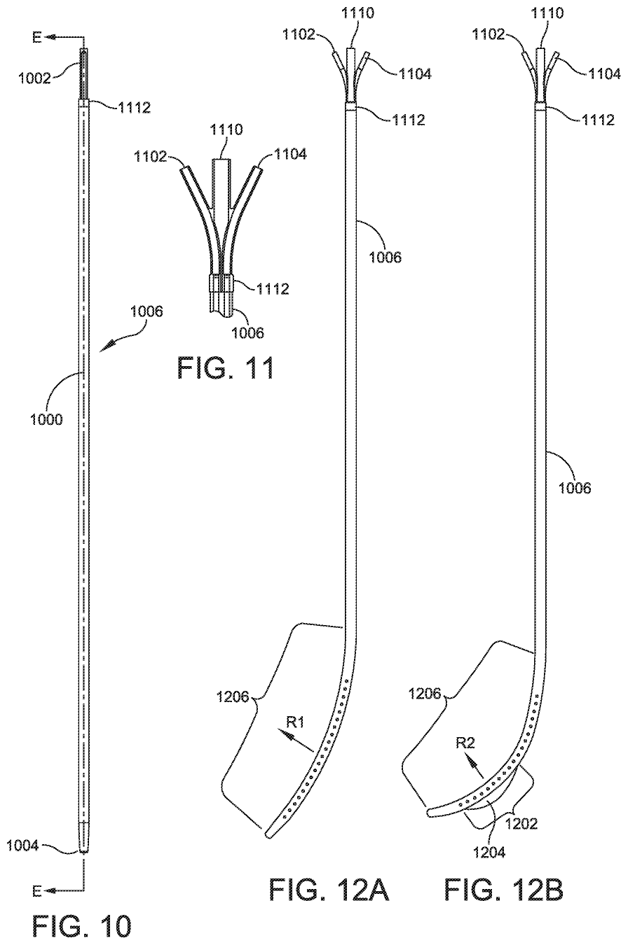

Turning now to FIG. 10, an alternative sleeve tube 1000 has an integral silicone main body 1006 with a proximal end 1002 opposite a distal end 1004. With reference to FIG. 11 a main channel 1110 and opposed balloon 1102 and sump 1104 channels feed together through a sealing sleeve 1112 into the main body section 1006. Conversely, the opposed balloon 1102 and sump 1104 channels extend upwardly from the sealing sleeve 1112 and bend away spaced from each other 1102, 1104, providing a spread and forked configuration of the ends of main channel 1110, balloon channel 1102, and sump channel 1104 opposite the sealing sleeve 1112. The width of this spread and forked configuration can make it impossible for the proximal end 1002 of the sleeve tube 1000 to penetrate the patient's mouth.

Referring to FIG. 12A, the sleeve tube 1000 assembly is formed to have a predetermined curved shape by placing the sleeve assembly 1000 in a curing cavity (not shown) having a predetermined curved section causing the curvable working section 1206 to curve within the cavity at a radius R1. Heat or another curing agent is then applied, causing the working section 1206 to bias toward taking on the predetermined shape of radius R1 when working section 1206 is subject to certain temperatures, such as when in the stomach of a patient. Depending on the materials used and the temperature of the working environment, the curvable working section 1206 can resume a straightened form when the sleeve tube 1000 is removed from the cavity and cools to room temperature, and then similarly return to having the pre-determined curvature when the curvable working section 1206 reaches the pre-determined temperature inside a patient's stomach.

With reference to FIG. 12B, when the sleeve tube and balloon 1000 is then inserted in a patient's stomach and the balloon 1204 is inflated by injecting gas into balloon channel 1102, the inflating balloon 1204 forces the adjacent portion 1202 of the curvable working section 1206 to curve more that the predetermined curvature provided by heating of the working section 1206 within the stomach. As result, the inflated balloon 1204 forces the adjacent portion 1202 of the working section 1206 to have a radius R2 smaller than, as shown in FIG. 12A, radius R1.

In one embodiment, the sleeve tube 1000 is made from silicone having a Shore hardness on the A scale of 25 to 30. The silicone can be, for example, SILASTIC® brand biomedical grade Liquid Silicone Rubber (LSR) from Dow Corning or Thermoset Elastomer (TSE), such as Dow Corning SILASTIC® 7-4860 BIO LSR (heat cured) or Dow Corning SILASTIC® Q7-4535 BIO ETR Elastomer (peroxide cured)). In some embodiments, the silicone or other material should be of medical grade, have maximum lubricious characteristics, and be directly bondable.

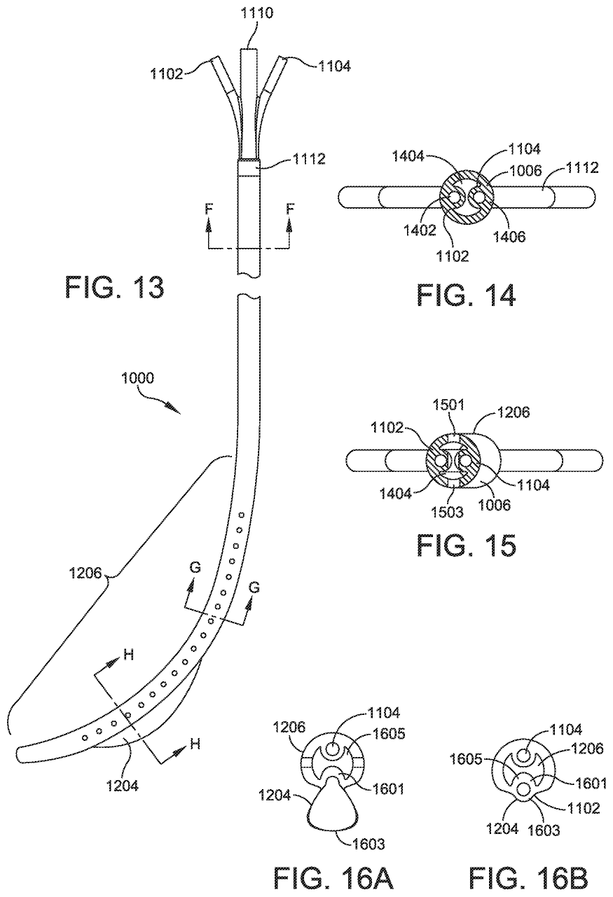

FIG. 13 is a partial elevational view of the sleeve tube 1000 of FIG. 10 with the working section 1206 in a curved configuration. With reference to FIG. 14, the opposed balloon channel 1102 and sump channel 1104 abut opposed interior sides of the interior wall of the main body section 1006. The remaining space 1404 within the of the interior of main body section 1006 provides a main body section channel 1404 in communication with, as shown in FIG. 13, the upwardly extending main channel 1110. In other words, with reference back to FIG. 14, the main body section 1006 has three interior, axially extending lumens 1402, 1404, and 1406 in communication with, as shown in FIG. 14, the balloon channel 1102, the main channel 1110, and the sump channel 1104, respectively. The balloon channel 1102 and sump channel 1104 abut the opposed sides of the internal periphery of the main channel 1110 so that the exterior periphery of the main body section 1006 may be tubular and thus enable easier insertion and bending of the sleeve tube 1000 during insertion into the body.

With reference now to FIG. 15, the working section 1206 in sleeve tube 1000 has laterally extending rows (not shown in FIG. 15) of tubular, radially extending perforations or passages 1501, 1503 passing from the exterior periphery of the working section 1206 into the main body section channel 1404. Gas and material can pass through these radial passages 1501, 1503.

With reference now to FIGS. 16A and 16B, the lower end of the working section 1206 provides a laterally extending balloon channel 1601 having a relatively thin outer wall 1603 as compared to the inner wall 1605 of the balloon channel 1102. The opposed portions of the main channel 1006 are also thinned at this location. Then, as shown in FIG. 16B, the relatively thin walled section 1603 provides the exterior wall of the balloon 1605 in its collapsed state. The thin walled section 1603 balloon can then, as shown in FIG. 16A, be blown up as shown in FIG. 16A and then subsequently, as shown in FIG. 16B, again be deflated to its collapsed state.

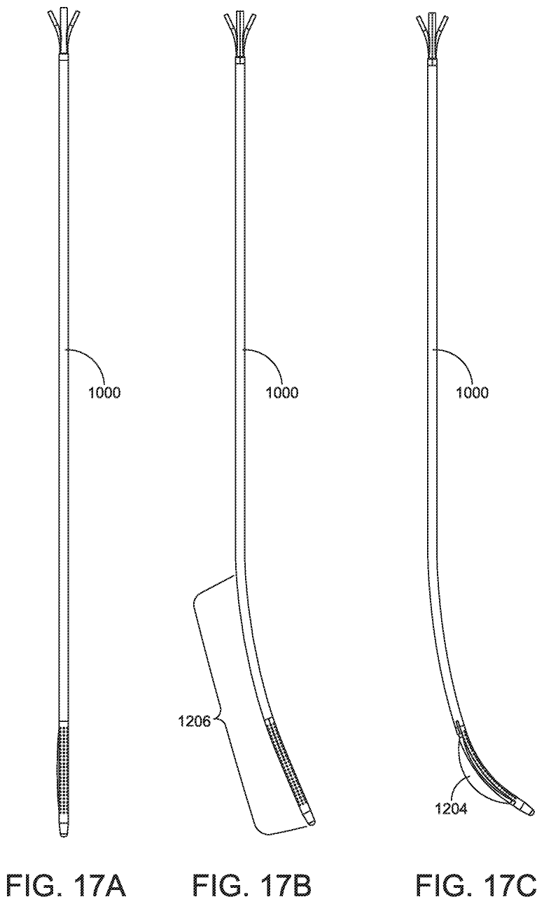

With reference to FIGS. 17A, 17B, and 17C, the sleeve tube 1000 may be fabricated as a single part multilayer co-extrusion. With reference to FIG. 17A, a blank of the entire sleeve tube 1000 may be extruded from multiple thermoplastic elastomers with different glass transition temperatures. Then, with reference to FIG. 17B, the entre blank can then be heated to glass transition temperature, with the working section 1206 thermoformed to create the curvature and perforations as shown in FIG. 17B. Subsequently, the now-thermoformed sleeve tube can be heated to the glass transition temperature for the balloon channel 202 in order to secure the balloon 116 to the balloon channel slot or wall 403 (not shown in FIG. 17B). The entire device may be sealed in ways well known in the art.

In use, the working section 1206 is straightened and inserted into the patient's mouth, esophagus, and stomach. Within the stomach, the working section 1206 returns to its free, curved state as in FIG. 17B. Then, with reference to FIG. 17C, the balloon 1605 may be inflated by the practitioner to cause the working section 1206 abutting the balloon 1605 to further curve within patient's stomach. Conversely, the balloon 1605 may be deflated to cause thee working section 1206 to return to its free state and allow the sleeve tube 1000 to be withdrawn from the patient. During withdrawal, the flexible working section 1206 flexibly straightens, as in FIG. 1, to pass through the esophagus without causing edema to the patient.

On reading this specification, those of skill in the art will recognize that many of the components discussed as separate units may be combined into one unit and an individual unit may be split into several different units. Further, the various functions could be contained in one computer or spread over several networked computers and/or devices. The identified components may be upgraded and replaced as associated technology improves, advances are made in computing technology, or based on a developers skills or preferences.

The process parameters, functions, system features, and sequence of steps described and/or illustrated herein are given by way of example only and may be varied and mixed and matched as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The foregoing detailed description has described some specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems, their components, and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.” Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and B and C).

Finally, any ranges stated above include all sub-ranges within the range.

What we claim is:

1. An orogastric tube comprising:
a proximal end section;
a distal end section opposite the proximal end section, the distal end section having a flexible, resilient curved portion coupled to a tapered tip;
a central conduit section extending from the proximal end section to the distal end section, the central conduit having:
(i) an outer periphery surrounding the central conduit;
(ii) a central conduit axis extending within the periphery;
(iii) a sump channel extending from the proximal end section to the distal end section; and
(iv) a suction channel extending from the proximal end section to the distal end section, wherein the curved portion of the distal end section resiliently bends away from the central conduit axis with the tapered tip having a tip axis at an angle to the central conduit axis.

2. The orogastric tube of claim 1, wherein the distal end section has a plurality of perforations penetrating the outer periphery of the distal end section in fluid communication with the main channel.

3. The orogastric tube of claim 2, wherein the orogastric tube further comprises the sump channel and the suction channel within the outer periphery enclosing a pre-determined length of the sump channel and suction channel.

4. The orogastric tube of claim 1, wherein the sump channel penetrates the distal end section and is in fluid communication with the outer periphery.

5. The orogastric tube of claim 3, wherein the sump channel penetrates the distal end section and is in fluid communication with outer periphery and the plurality of perforations.

6. The orogastric tube of claim 1, wherein the sump channel penetrates the distal end section and is in fluid communication with the outer periphery and the plurality of perforations.

7. The orogastric tube of claim 2, wherein the sump channel penetrates the distal end section and is in fluid communication with the main channel and the plurality of perforations.

8. The orogastric tube of claim 3, wherein the sump channel penetrates the distal end section and is in fluid communication with the outer periphery.

9. An orogastric sleeve tube comprising:
a proximal end section;
a curved distal end section opposite the proximal end, the curved distal end section coupled to a tapered tip;
a multichannel, laterally extending tubular body housing, an inner volume of the housing fully surrounding a suction channel, and an outer surface of a sump channel extending along the inside of the lateral length of the of the multichannel, laterally extending tubular body;
a working section in the distal end section and having material transfer perforations penetrating the outer periphery of the working section in material transfer communication with the main channel and sump channel.

10. The orograstric sleeve tube of claim 9 further comprising a curved section of predetermined radius in the distal end section.

11. The orogastric sleeve tube of claim 9 further comprising an aperture section in the proximal end section and providing a suction channel aperture and sump channel aperture.

12. The orogastric sleeve tube of claim 10 further comprising a widened aperture section in the proximal end section and providing a suction channel aperture and a sump channel aperture, the width of the widened aperture section being wider than the width of the multichannel, laterally extending tubular body housing.
